(19) United States
(12) Patent Application Publication
(10) Pub. No.: US 2009/0131960 A1
(43) Pub. Date: May 21, 2009

(54) TONGUE CLEANER

(75) Inventor: Michio Tanaka, Niigata-shi (JP)

Correspondence Address: DARBY & DARBY P.C., P.O. BOX 770, Church Street Station, New York, NY, 10008-0770, US

(73) Assignees: Shikien Co., Ltd., Niigata-shi (JP); Mikio Yuzawa, Tokyo (JP)

(21) Appl. No.: 12/196,098

(22) Filed: Aug. 21, 2008

(30) Foreign Application Priority Data
Feb. 6, 2007 (JP) 2007-026614

(51) Int. Cl. A61B 17/24

(52) U.S. Cl. 606/161

(57) ABSTRACT

The present invention aims at providing a tongue cleaner which can be used with one hand without damaging an inside surface of a roof of the mouth. A tongue cleaner of the present invention comprises a head 1, and a rod-shaped handle 3 connected to one end of the head 1. The tongue cleaner includes, on either its front or its back face, a flat and thin head with a concavely curved face having a valley portion extending parallel to a longitudinal direction of the rod-shaped handle, and a woven textile 4 having a roughened surface, serving as a sheet having a porous or multi-gap structure, attached to the concavely curved face 1a. When cleaning both sides of a tongue, the concavely curved face 1a of the head 1 is used. The head 1 can be moved back and forth while pressing it against a tongue, without causing any concerns for possible lateral displacement of the head on the surface of the tongue and without causing nausea during the cleaning.

### TECHNICAL FIELD

The present invention relates to a tongue cleaner suitably applicable to cleaning a tongue.

### BACKGROUND OF THE INVENTION

Heretofore, there have been proposed tongue cleaners in Japanese unexamined patent application publication No. 2001-161720, Japanese utility model registration publication No. 2515465, etc. The tongue cleaner disclosed in the former publication comprises a head, a neck connected to the head, and a handle connected to the neck. The head is formed flat and thin, having plural brush bodies protruded from the surface thereof such that the brush bodies are disposed at desired intervals in a straight line or in an arc in the direction orthogonal to the longitudinal direction of the handle. The top surface of each brush body is formed such that the contour defined by the top surfaces of the brush bodies is formed symmetrical as a whole so as to mate with the surface of the section of a tongue. The tongue cleaner disclosed in the latter publication is the one in which a looped pile textile at least pile portion of which is made up of multifilament yarns is fixed to a flexible stick-like member.

Further, U.S. Pat. No. 6,171,323 discloses, as another conventional art, a tongue cleaner comprising an elongate edge portion, being contoured over its length to have a relatively deep central portion for scraping the tongue; an elongate shank portion; and a supporting portion extending from the shank to support the edge portion.

Furthermore, U.S. Pat. No. 5,984,935 discloses another tongue cleaner comprising a head including a base portion constituted by a frame defining a substantially oval opening and having an upper face and a lower face, and a plurality of curved blades formed with active scraping surfaces and at least partly projecting from said upper face.

Moreover, U.S. Pat. No. 6,019,773 discloses another tongue cleaner comprising a set of absorbent core members that are flexible but capable of retaining their shapes; and a portion between the core members that is flexible and indented and defines a lateral centerline.

The head of the tongue cleaner according to the above-mentioned Japanese unexamined patent publication No. 2001-161720 has an enough width to cover substantially the whole width of a tongue, and can be used without causing nausea when a center or back of a tongue is cleaned. However, in the case of cleaning a portion near the right or left side of a tongue, a user carries out the cleaning by lifting up a portion of the head opposite to the target side of a tongue to be cleaned, so that a rear face of the head at a lifted side may contact the inside surface of a roof of a user's mouth, thus leading to a likelihood of the user being hurt by the same. On the other hand, the tongue cleaner according to Japanese utility model registration publication No. 2515465 is the one in which a looped pile textile at least pile portion of which is made up of multifilament yarns is fixed to a stick-like member having flexibility but made of synthetic resin or metal. This stick-like member makes up a tongue coating scraping brush by fixedly bonding the loop pile textile to the stick-like polyethylene member (thin-plate-shaped, spatula-shaped or pipe-shaped) that is 2 mm in thickness, 10 mm in width, and 230 mm in length, with both ends of the stick-like member remaining free from the textile up to a 40 mm length, respectively. Hence, both the ends of the stick-like member where the loop pile textile is not fixedly bonded can be pinched between thumbs and index fingers of both hands. As a result, it is difficult to clean a back of tongue, and what is more, it is very hard to use with one hand.

Moreover, according to the conventional art disclosed in U.S. Pat. No. 6,171,323, the edge portion of the tongue cleaner for scraping tongue coat off is tabular or plate-like, so that there may have difficulties in scraping tongue coat.

Still moreover, according to the conventional art disclosed in U.S. Pat. No. 5,984,935, the edge portion has a linear shape, causing concerns that a contact area of the edge portion may become too small.

Furthermore, according to the conventional art disclosed in U.S. Pat. No. 6,019,773, it is hard to scrape a back of tongue because the cleaner is put over a finger in use.

The present invention aims at providing a tongue cleaner which can be used with one hand only, without damaging an inside surface of a roof of the mouth. The present invention also aims at providing a tongue cleaner enabling tongue coating to be reliably removed from the tongue.

### SUMMARY OF THE INVENTION

A tongue cleaner according to the present invention comprises a head and a rod-shaped handle connected to one end of the head. The tongue cleaner includes, on either a front or back face thereof, a flat and thin head with a concavely curved face having a valley portion extending parallel to a longitudinal direction of the rod-shaped handle, and a sheet with a roughened surface having a porous or multi-gap structure, attached to the concavely curved face.

The head may be formed in a flat and thin shape having substantially even thickness, including, on either its front or back face, the concavely curved face having the valley portion extending parallel to the longitudinal direction of the rod-shaped handle while the other side of the head may be formed in a convexly curved shape. Alternatively, the sheet may be attached to both of the front and back faces, or a woven textile or a nonwoven textile may be used as the sheet.

Further, the foregoing woven textile may be the one in which either weft yarns or warp yarns are mainly raised so that either the raised weft yarns or raised warp yarns of the woven textile can be attached to either one or both of the front face and back face of the head in a direction intersecting with the longitudinal direction of the rod-shaped handle. Alternatively, the woven textile may be a pile-woven textile, in which pile yarns are used for the weft yarns or warp yarns of the woven textile, and the pile yarns may be attached in a direction intersecting with the longitudinal direction of the rod-shaped handle.

When cleaning tongue coatings at both sides of a tongue using this tongue cleaner, protuberant sides of the tongue are both allowed to be well fitted in the above-mentioned concavely curved face, and thus the tongue cleaner is free from lateral displacement during the use so that the tongue coating is cleaned simply by grabbing and moving the rod-shaped handle back and forth with said concavely curved face in contact with the tongue.

### BRIEF DESCRIPTION OF THE DRAWINGS

FIG. 1 is a top plan view of a tongue cleaner according to an embodiment of the present invention.

FIG. 2 is a sectional view thereof taken on a line A-A of FIG. 1.

FIG. 3 is an enlarged sectional view thereof taken on a line B-B of FIG. 1.

FIG. 4 is an enlarged sectional view of a main part of a tongue cleaner, illustrating a head thereof according to a modified example of an embodiment of the present invention.

FIG. 5 is an enlarged sectional view of a main part of a tongue cleaner, illustrating a head thereof according to another modified example of an embodiment of the present invention.

FIG. 6 is a top plan view of a main part of a tongue cleaner, illustrating a head thereof according to another modified example of an embodiment of the present invention.

FIG. 7 is a structural diagram of a plain-woven textile attached to the front and back faces of the head of the tongue cleaner of the present invention.

FIG. 8 is a sectional view taken on a line C-C of FIG. 7 according to the present invention.

FIG. 9 is a structural diagram of a twill-woven textile attached to the front and back faces of the head of the tongue cleaner of the present invention.

FIG. 10 is a sectional view taken on a line D-D of FIG. 9 according to the present invention.

FIG. 11 is a structural diagram of a stain-woven textile attached to the front and back faces of the head of the tongue cleaner.

FIG. 12 is a sectional view taken on a line E-E of FIG. 1 according to the present invention.

FIG. 13 is a structural diagram of a pile-woven textile attached to the front and back face of the head of the tongue cleaner.

FIG. 14 is a perspective view illustrating a tongue cleaner according to another embodiment of the present invention.

FIG. 15 is a top plan view of a main part of the tongue cleaner thereof.

FIG. 16 is a sectional view taken on a line F-F of FIG. 15 according to the present invention.

FIG. 17 is a sectional view taken on a line G-G of FIG. 15 according to the present invention.

FIG. 18 is an enlarged sectional view of a main part according to another embodiment of the present invention.

FIG. 19 is an explanatory diagram illustrating the tongue clearer in use.

### DETAILED DESCRIPTION OF THE INVENTION

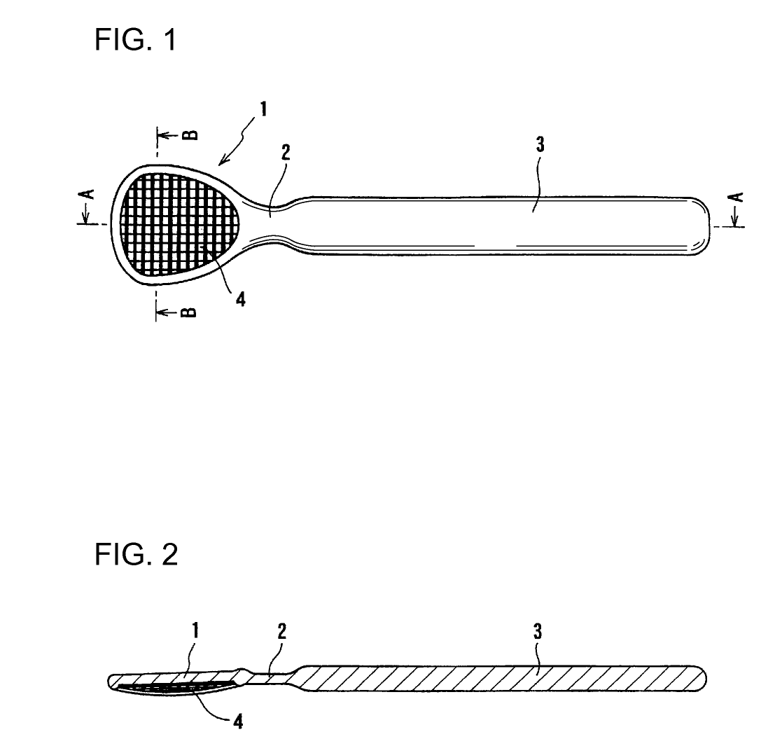

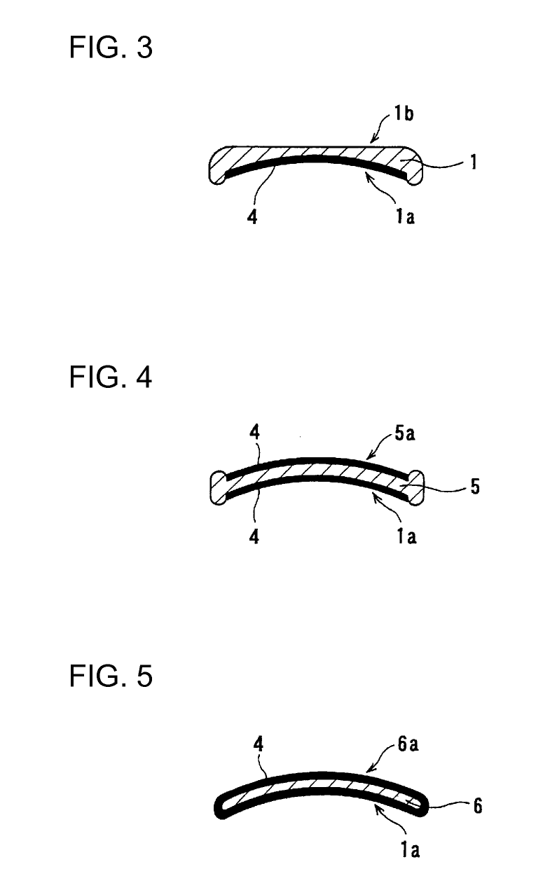

As shown in FIG. 1 through FIG. 3, a tongue cleaner according to the present invention comprises a head 1, a neck 2, and a rod-shaped handle 3. The head 1 and the rod-shaped handle 3 are connected with each other via the neck 2 having flexibility and a narrow shape. This head 1 is formed in a flat-and-thin shape and has one face and a reverse face. The one face includes a concavely curved face 1a having a valley portion extending parallel to a longitudinal direction of the rod-shaped handle 3, and the reverse face opposite to the first face is formed into a substantially flat face 1b. The rod-shaped handle 3 is formed in a shape capable of being easily grasped with a hand. A woven textile 4 acting as a sheet is attached to the concavely curved face 1a of the head 1. The details of the woven textile 4 will be described later. It is desirable to attach the woven textile 4 on the substantially flat reverse face opposite to the head 1. The methods for attaching the woven textile 4 to the head 1 include a bonding method by means of an ordinal adhesive material or a pressure sensitive adhesive material, or a bonding method utilizing high-frequency wave or ultrasonic wave to thereby fixedly join the woven textile 4 to the head 1. In the meantime, whilst a variety of the woven textiles 4 are illustrated and described with reference to the drawings shown in connection with respective embodiments of the invention, the sheet of the invention is not limited to the illustrated woven textiles 4, but it may be any flexible sheet having a porous or multi-gap structure such as a nonwoven sheet (nonwoven paper in general) or a foamed sheet, having a roughened surface with numerous fine concavities and convexities formed thereon. Alternatively, a flexible sheet with textiles or hairs implanted thereon may also be used.

According to the tongue cleaner with the above configuration, a user has only to take the following steps to clean a tongue: grasping the rod-shaped handle 3 with a hand to softly press the woven textile 4 of the head 1 onto any one of right and left upper faces in a tongue; and then, moving the hand with the rod-shaped handle 3 back and forth along the longitudinal direction of the rod-shaped handle 3. Further, even if the head 1 is pressed to a portion near the right or left side of a tongue, the head 1 is less likely to be displaced sideways to disengage from the upper face of the tongue. In this way, when the head 1 is moved back and forth with the woven textile 4 pressed on a tongue, numerous fine concavities and convexities on the surface of the woven textile 4 can scrape out dirt or grime while raising the tongue coating on the tongue and drawing out the dirt or grime hidden behind the tongue coating. For a center or back of a tongue, a user may clean such section using the woven textile 4 attached to the flat face 1b on the reverse face of the head 1.

Moreover, the head 1 is formed in a flat-and-thin shape, so that both lateral sides of the head 1, which are the thickest sections, are as thin as about 8 mm at most. Therefore, even if the woven textile 4 of 1 mm thickness is attached to both the top and rear faces of the head 1, total thickness of the head 1 will be as small as about 10 mm or less (general toothbrushes are about 15 mm in thickness). As described above, even though the tongue cleaner, about ⅔ as thick as general toothbrushes, is moved back and forth in a mouth cavity, it can be used without causing nausea during the cleaning. Moreover, in the case of cleaning a center or back of a tongue, the woven textile 4, attached to the substantially flat reverse face 1b of the head 1, may be pressed to a tongue. In that case, however, it is more effective to use the woven textile 4 attached to the convexly curved face that is shown in a hereinafter-described embodiment.

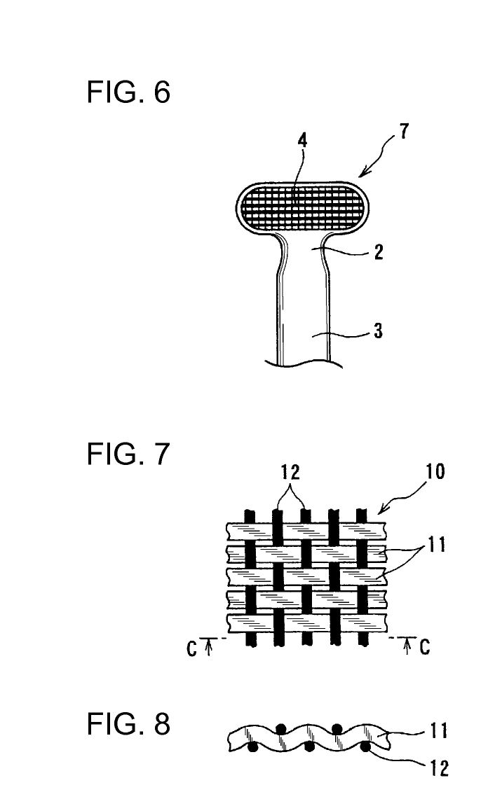

Next is a description of an example illustrated in FIG. 4. Duplicate description of the same parts as those described in the foregoing example will be omitted and only different features will be mainly described (the same is applied to hereinafter-described embodiments). The tongue cleaner includes a head 5 that is formed with the concavely curved portion 1a on one side of the head 1 and a convexly curved face 5a on a reverse side by bending the whole of the head 1 along the direction intersecting with the longitudinal direction of the rod-shaped handle 3. In the meantime, whilst the woven textile 4 is attached to both the front and reverse sides of the head 5 in the foregoing examples, the woven textile 4 may cover the whole of a head 6, as illustrated in FIG. 5. In that case, there is only a slight difference in effect between both the examples. Reference symbol 6a denotes a convexly curved face as a reverse face of the head 6. Furthermore, although a planar shape of the head 1 is described as a deformed elliptical shape in the foregoing description, other shapes may be employed such as a horizontally elongated oval head 7 as illustrated in FIG. 6.

According to the tongue cleaner with the above configuration, it is possible to clean center and back of a tongue in addition to the effect described in the foregoing embodiment, by pressing the woven textile 4 attached to the convexly curved faces 5a, 6a of the heads 5, 6 against the upper face of the center or back of a tongue, which is a concavely curved face of a tongue, and then by moving the rod-shaped handle 3 grasped with a hand back and forth along the longitudinal direction of the rod-shaped handle 3. In the meantime, when cleaning a center or back of a tongue, the woven textile 4, attached to the flat face 1b of the head 1, may be pressed to a tongue. In that case, however, it is more effective to use the woven textile 4 attached to the convexly curved face that is shown in a hereinafter-described embodiment.

A further detailed description is given to the woven textile 4 attached to both of the front and reverse faces of the heads 1, 5, 6, and 7 described in the above embodiment. FIG. 7 is a structural diagram of a plain-woven textile, and FIG. 8 is a sectional view on a line C-C of FIG. 7. In the drawings, weft yarns 11 are illustrated as being thicker than warp yarns 12. As illustrated in FIG. 8, in a woven textile, warp yarns are straight. In contrast, weft yarns snake their ways so as to avoid the warp yarns. Therefore, if the weft yarns 11 thicker than the warp yarns 12 are used in the plain-woven textile 10, numerous fine concavities and convexities on a surface of the plain-woven textile 10 get larger and rougher. These weft yarns 11 are attached to the concavely curved face 1a, the flat face 1b, the convexly curved faces 5a, and 6a of the front and reverse faces of the heads 1, 5, 6, and 7 along the direction intersecting with the longitudinal direction of the rod-shaped handle 3 (desirably along an orthogonal direction). And then, by moving the heads 1, 5, 6, and 7 pressed onto the surface of the tongue, numerous fine concavities and convexities on the surface of the plain-woven textile 10 function to raise tongue coatings to make it easier to draw out dirt hidden behind the tongue coating. In the meantime, if the warp yarns 12 thicker than the weft yarns 11 are used for the plain-woven textile 10, these warp yarns 12 may be attached to the concavely curved face 1a, the flat face 1b, the convexly curved faces 5a, and 6a of the front and reverse faces of the heads 1, 5, 6, and 7 along the direction intersecting with the longitudinal direction of the rod-shaped handle 3.

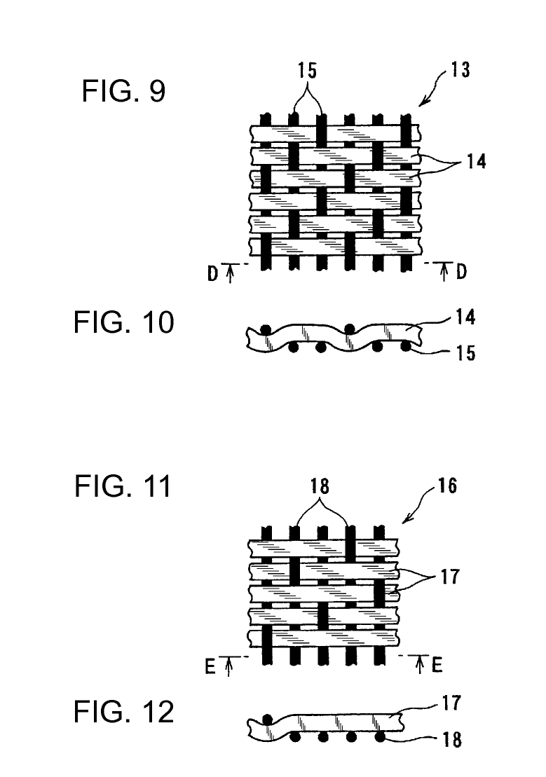

FIG. 9 is a structural diagram of a twill-woven textile 13, and FIG. 10 is a sectional view on a line D-D of FIG. 9. In these drawings as well, the weft yarns 14 are illustrated as thicker than the warp yarns 15. In the case of the twill-woven textile 13, the weft yarns 14 are woven as stepping over every two of the warp yarns 15 and are uplifted on the surface of the twill-woven textile 13, so that the surface of the twill-woven textile 13 makes tongue coating easy to scrape. Thus, there can be obtained a larger effect of scraping off the dirt hidden behind the tongue coating. As shown in FIG. 9 and FIG. 10, if the weft yarns 14 are formed thicker than the warp yarns 15, it is even more effective. These weft yarns 14 are attached to the concavely curved face 1a, the flat face 1b, the convexly curved faces 5a, and 6a of the first and the second faces of the head 1, 5, 6, and 7 along the direction intersecting with the longitudinal direction of the rod-shaped handle 3 (desirably along an orthogonal direction), and then, by moving the heads 1, 5, 6, and 7 pressed onto the surface of the tongue back and forth, the weft yarns 14 uplifted on the surface of the woven textile function to raise tongue coatings to make it easier to draw out dirt hidden behind the tongue coating. In the meantime, if the warp yarns 15 thicker than the weft yarns 14 are used, the warp yarns 15 may be attached to the concavely curved face 1a, the flat face 1b, the convexly curved faces 5a, and 6a of the front and reverse faces of the heads 1, 5, 6, and 7 along the direction intersecting with the longitudinal direction of the rod-shaped handle 3.

FIG. 11 is a structural diagram of a stain-woven textile 16, and FIG. 12 is a sectional view on a line E-E of FIG. 11. In these drawings as well, the weft yarns 17 are illustrated as being thicker than the warp yarns 18. In the case of the stain-woven textile 16, the weft yarns 17 are woven as stepping over every four or more of the warp yarns 18 and are uplifted on the surface of the stain-woven textile 16, so that the surface of the stain-woven textile 16 makes tongue coating easy to scrape. Thus, there can be obtained an even larger effect of scraping off the dirt hidden behind the tongue coating than the twill-woven textile 13. If the weft yarns 17 are formed thicker than the warp yarns 18, it is even more effective. These weft yarns 17 are attached to the concavely curved face 1a, the flat face 1b, the convexly curved faces 5a, and 6a of the front and the reverse faces of the heads 1, 5, 6, and 7 along the direction intersecting with the longitudinal direction of the rod-shaped handle 3 (desirably along an orthogonal direction), and then, by moving the heads 1, 5, 6, and 7 pressed onto the surface of a tongue back and forth, the weft yarns 17 uplifted on the surface of the stain-woven textile 16 function to raise tongue coatings to make it easier to draw out dirt hidden behind the tongue coating. In the meantime, if the warp yarns 18 thicker than the weft yarns 17 are used, the warp yarns 18 may be attached to the concavely curved face 1a, the flat face 1b, the convexly curved faces 5a, and 6a of the front and reverse faces of the heads 1, 5, 6, and 7 along the direction intersecting with the longitudinal direction of the rod-shaped handle 3.

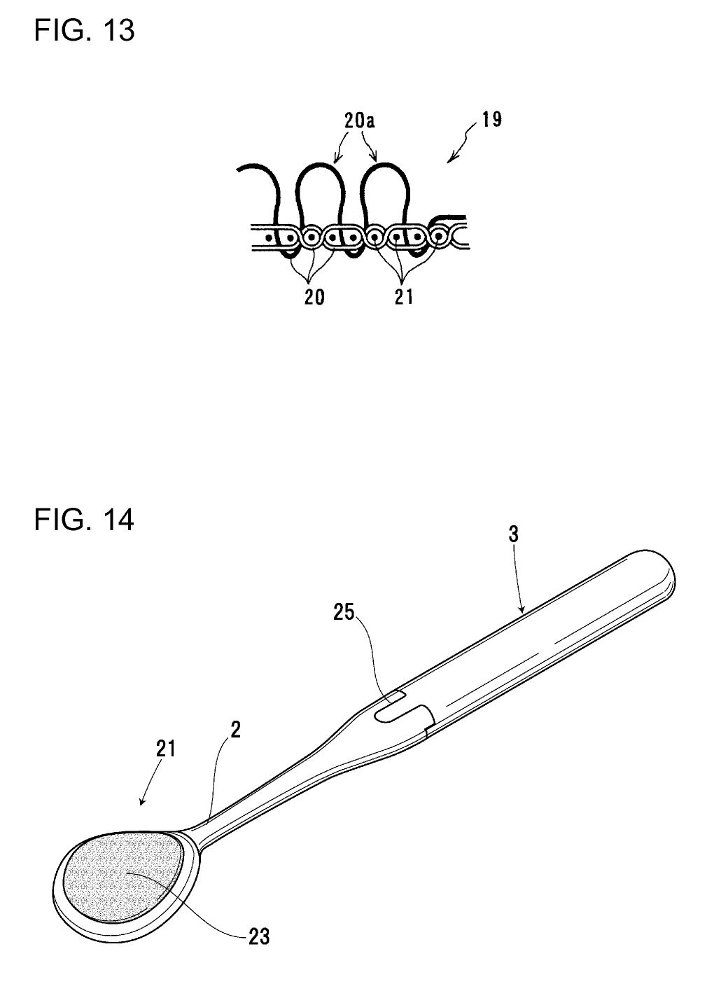

FIG. 13 is a structural diagram of a pile-woven textile 19. In the case of the pile-woven textile 19, it is woven so as to project part (i.e., pile yarns) of warp yarns 20 comprising pile yarns and ground warp yarns from the surface of the pile-woven textile 19, as illustrated in the drawing as ring-like loops 20a. As a result, these loops 20a of the pile yarns 20 function to raise tongue coating as these loops 20a get entangled with the tongue coating, making it easier to scrape off the dirt behind the tongue coating, thus achieving an even more excellent effect. The numerous loops 20a of the pile yarns 20 protruded from the surface of the pile-woven textile 19 are attached to the concavely curved face 1a, the flat face 1b, the convexly curved faces 5a, and 6a of the front and reverse faces of the heads 1, 5, 6, and 7 along the direction intersecting with the longitudinal direction of the rod-shaped handle 3 (desirably along an orthogonal direction), and then, by moving the heads 1, 5, 6, and 7 pressed onto the surface of the tongue back and forth, the numerous loops 20a of the pile yarns 20, as the warp yarns uplifted on the surface of the pile-woven textile 19, are allowed to get entangled with the tongue coating, making it easier to scrape off the dirt behind the tongue coating. In the meantime, reference numeral 21 denotes weft yarns.

As described above, a tongue cleaner according to the foregoing embodiment comprises the head 1 and the rod-shaped handle 3 connected to one end of the head 1. The tongue cleaner includes, on either its front or its back face, the flat and thin head 1 with the concavely curved face 1a having a valley portion extending parallel to a longitudinal direction of the rod-shaped handle 3, and the porous or multi-gap sheet, acting as the woven textile 4, having a roughened surface, attached to the concavely curved face 1a. When cleaning tongue coating on a tongue at both upper sides of the tongue using this tongue cleaner, the tongue cleaner is free from lateral displacement because both protuberant sides of the tongue are caught in this concavely curved face 1a, so that the tongue coating is cleaned simply by grabbing the rod-shaped handle 3 and moving the rod-shaped handle 3 back and forth with the concavely curved face 1a in contact with the tongue. Furthermore, when cleaning tongue coating on the concavely curved face 1a, the reverse face of the head 1 does not contact the inside surface of a roof of the mouth despite the movement of the flat-and-thin head 1 toward back and front, so that the tongue coating can be cleaned smoothly. The width of this concavely curved face is about ½ of a tongue's width. Therefore, cleaning a tongue can be performed easily because the head 1 is not raised a lot in the case of cleaning either right or left side of a tongue. Moreover, the woven textile 4 attached to the concavely curved face 1a has a lot of fine concavities and convexities on the surface, leading to the roughened surface thereof. As a result, every time the head 1 is moved back and forth on a tongue by grasping the rod-shaped handle 3 with a hand, these concavities and convexities function to raise the tongue coating on the tongue, thus ensuring the cleaning of a tongue due to the dirt behind the tongue coating being scraped out. Additionally, fine concavities and convexities are soft because they are made of a woven textile or a nonwoven textile sheet, so that they do not hurt a tongue. As discussed from the above, in the case of cleaning not only the upper surface of but also both sides of a tongue using the tongue cleaner having the flat-and-thin head 1, tongue coating can be reliably cleaned without damaging the tongue or the inside of the mouth, and the tongue cleaner can be used without causing nausea during the cleaning.

Further, the head 5 includes, on either a front or reverse face, the concavely curved face 1a having a valley portion extending parallel to the longitudinal direction of the rod-shaped handle 3, and the head 5 is formed to a substantially even thickness and in a flat-and-thin shape, while the other side of the head 5 is formed in a convexly curved shape, thus advantageously reducing possibilities of hurting the inside of the mouth because the cleaner head is less likely to be uplifted from the center of a tongue when either side of the tongue is cleaned, in addition to the above-mentioned advantageous effect of the tongue cleaner.

Furthermore, in addition to the above effects, the convex reverse side of the head can more easily clean the slight concavity in the center and back of a tongue, according to the tongue cleaner having the sheet attached to both of the front and back faces of the head.

Moreover, according to the tongue cleaner of which the woven textile 4 acting as the sheet is a woven or nonwoven textile, the surface of the woven textile or the nonwoven textile is formed rough, so that the same effects as above respective tongue cleaners can be obtained.

Additionally, the tongue cleaner includes the woven textile called the twill-woven textile of which the weft yarns or the warp yarns are mainly raised, and the raised weft yarns or the warp yarns for the twill-woven textile are attached to either one or both of the front and reverse face of the head 1 in the direction intersecting with the longitudinal direction of the rod-shaped handle 3. Since these raised weft yarns 11 or the warp yarns 12 are provided in the direction intersecting with the longitudinal direction of the rod-shaped handle 3, when the head 1 is moved back and forth with the woven textile 4 pressed on a tongue, raised weft yarns or warp yarns can scrape off the dirt behind the tongue coating while raising the tongue coating on the tongue. Thus, cleaning a tongue gets even more easier.

In addition, the tongue cleaner has the pile-woven textile as the woven textile 19, in which the pile yarns are used as weft yarns or warp yarns for the pile-woven textile. The pile yarns are attached in the direction intersecting with the longitudinal direction of the rod-shaped handle 3. Since these pile yarns are in the direction intersecting with the longitudinal direction of the rod-shaped handle 3, when the head 1 is moved back and forth with the woven textile 4 pressed on a tongue, pile yarns can scrape and clean the dirt behind the tongue coating while raising the tongue coating on the tongue. Besides, the parts of the pile yarns protruded from the undelayment of the textile are long, cleaning of a tongue gets more effective.

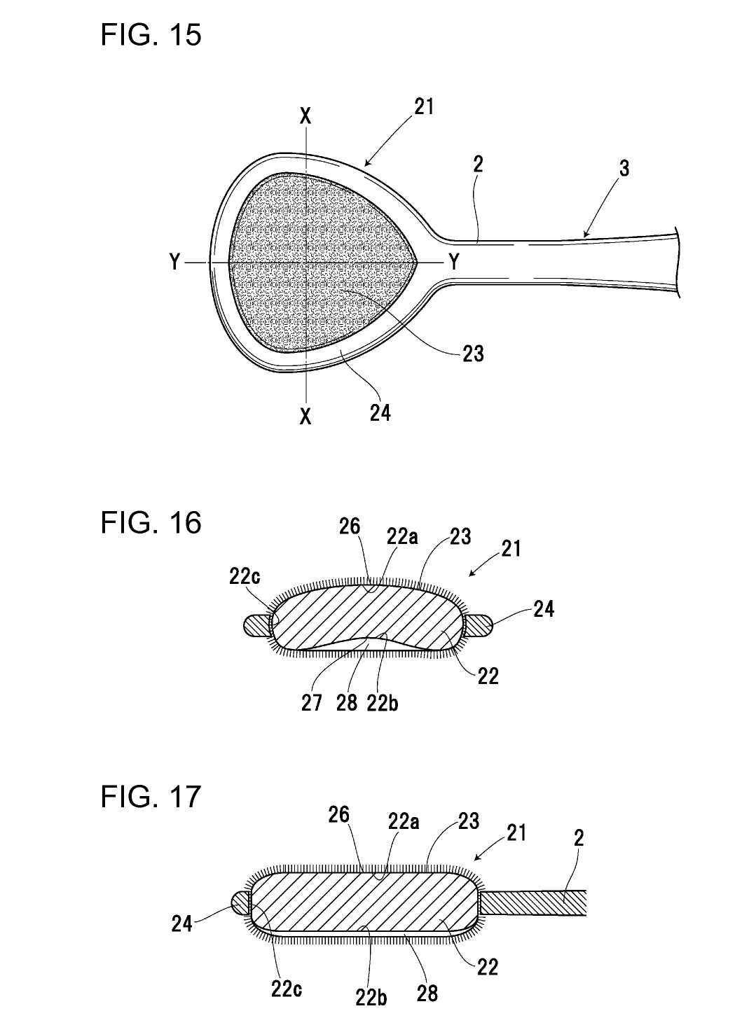

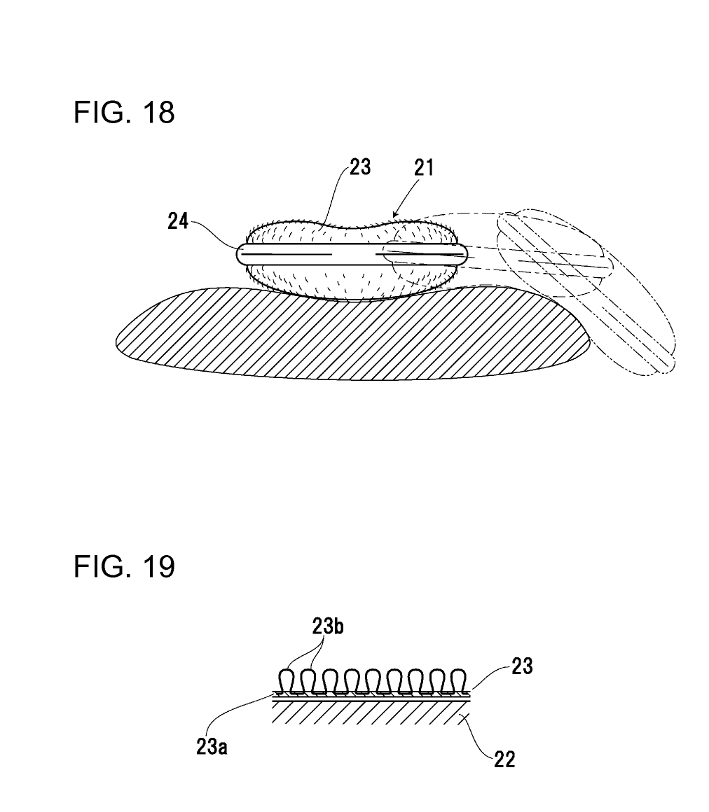

FIG. 14 through FIG. 19 illustrate another embodiment. Duplicate description of the same parts as those described in the above embodiment will be omitted. In the embodiment, a head 21 provided on a distal end of the rod-shaped handle 3 in a manner protruding forward includes a core member 22 made of hard resin, a textile 23 covering the surface of the core member 22 as described above, and a ring 24 that is connected to a distal end of the neck 2 and fixed outside through the textile 23. The rod-shaped handle 3 is divided into two parts, i.e., a front part and a back part, and the two parts can be connected through an insertion portion 25. The core member 22 has a flat surface, having a substantially isosceles triangular shape or equilateral triangular shape in plan view, with a comparatively large width at a distal side and a comparatively small width at a neck side. In this embodiment, the core member 22 is formed in a substantially equilateral triangular shape with rounded corners.

For one of flat surfaces of the core member 22, namely, for an upper face thereof, a convexly curved face 22a is formed so that an apex part 26 is provided along the longitudinal direction of the rod-shaped handle 3 so as to be contoured to fit the surface of a tongue. For the other of the flat surfaces of the core member 22, namely, for the lower face thereof, a concavely curved face 22b is formed so that a valley part 27 is provided along the longitudinal direction of the rod-shaped handle 3 so as to be contoured to fit the surface of a tongue. Then, the textile 23 is provided so as to substantially come in close contact with the convexly curved face 22a and the concavely curved face 22b, respectively. The textile 23 is one in which fibers 23b are protruded toward the outside on the surface of a flat main body of the textile 23a. In this embodiment is shown a case where a pile-woven textile is used.

The core member 22, the textile 23, and the ring 24 are integrated with one another by allowing the textile 23 to cover the core member 22 in a tensioned state, and fixedly fitting the ring 24 from the outside to respective side surfaces 22c located at the distal, neck and lateral sides of the core member 22. This integration is carried out in such a manner that the textile is allowed to cover the core member 22 in a tensioned state, and then, both of them are placed in a cavity (not illustrated) inside a mold for synthetic resin molding to fill up molten resin in a space outside the textile 23. In the meantime, the ones integrated inside the mold in this way can be taken out by opening the mold. Furthermore, the ring 24 is linearly provided in the center on the side face of the core member 22 along a peripheral side surface thereof. This ring 24 is arranged annularly in a plan view, in the form of a comparably thin line along the side of the core member 22 in a comparably thin line, having a thickness smaller than that of the core member 22 so as not to disturb the cleaning operation in a mouth.

In this way, in the state in which the core member 22, the textile 23 and the ring 24 are integrated with one another, the textile is fixed by the ring 24 at the distal, neck, right and left sides of the side faces 22c. Therefore, the other sides or parts of the textile are made slidable relative to the surface of the core member 22 while they are being kept in a tensioned state. Specifically, at the convexly curved face 22a of the core member 22, the upper face of the textile 23 is slidably in close contact with the face 22a. On the other hand, at the concavely curved face 22b of the core member 22, the lower face of the textile 23 is also slidable but not in close contact therewith though it is still kept in a tensioned state, so as to be slightly uplifted with the valley part 27 and a gap 28 formed therebetween.

Accordingly, as shown in FIG. 18, in the case of grasping the rod-shaped handle 3 with a hand and inserting the head 21 into the mouth to use it on a valley section of a tongue, tongue coatings are scraped off by allowing the head 21 to contact a tongue with the convexly curved face 22a directed downward as illustrated in a full line. In this case, a portion of the textile 23, which is located outside and opposed to the convexly curved face 22a of the core member 22, is slightly slidable thereto, so that if a force that is large to a certain extent is applied to the rod-shaped handle 3, yet the force will not immediately transmit to the textile 23 from the core member 22. Thus, possibilities to hurt a tongue can be reduced due to the reduction of the force by the sliding.

On the other hand, in the case of grasping the rod-shaped handle 3 with a hand and inserting the head 21 into the mouth to use it on a convex section such as a corner of a tongue, tongue coatings are scraped off by allowing the head 21 to contact a tongue with the concavely curved face 22b directed downward as illustrated in a dashed line or a dashed-two dotted line. In this case, a portion of the textile 23, which is located outside and opposed to the concavely curved face 22b of the core member 22, is slightly slidable with the gap 28 therebetween, so that if a force that is large to a certain extent is applied to the rod-shaped handle 3, yet the force will not immediately transmit to the textile 23 from the core member 22. Thus, possibilities to hurt a tongue can be reduced due to the reduction of the force by the sliding.

Thus, in the head 21, the textile 23 opposed to the concavely curved face 22b is allowed to slide relative to the same because the textile 23 serving as the sheet is provided on the surface of the core member 22 with the concavely curved face 22b formed thereon, while the textile 23 is fixed at the sides of the core member 22. Therefore, the textile 23 is allowed to contact a tongue softly, and if a certain degree of large force is applied to the rod-shaped handle 3, yet the force will be eased due to the sliding of the textile 23.

Moreover, since the gap 28 is formed between the concavely curved face 22b and the portion of the textile 23 opposed thereto, the textile 23 becomes easier to slide.

Still moreover, in the head 21, the textile 23 opposed to the convexly curved face 22a is allowed to slide relative to the same because the textile 23 is provided on the surface of the core member 22 with the convexly curved face 22a formed thereon, while the textile 23 is fixed at the sides of the core member 22. Therefore, the textile 23 is allowed to contact a tongue softly, and if a certain degree of large force is applied to the rod-shaped handle 3, yet the force will be eased due to the sliding of the textile 23.

Besides, the head 21 is configured such that other portions of the textile 23 than the side portions thereof are allowed to slide with the portions of the textile 23 at the sides of the core member 22 being fixed, since the textile 23 covers the surface of the core member 22 having a flat cross section, while the ring 24 connected to the rod-shaped handle 3 is fitted from outside to the sides of the textile.

What is claimed is:

1. A tongue cleaner, comprising:
a head; and
a rod-shaped handle connected to one end of said head,
wherein said head is formed flat and thin, provided, on either a front or back face thereof, with a concavely curved face having a valley portion extending parallel to a longitudinal direction of the rod-shaped handle, and a sheet with a roughened surface having a porous or multi-gap structure is attached to said concavely curved face.

2. The tongue cleaner according to claim 1, wherein said head is formed in a flat and thin shape having substantially even thickness, including, on either its front or back face, said concavely curved face having the valley portion extending parallel to the longitudinal direction of said rod-shaped handle, while an other side of said head is formed in a convexly curved shape.

3. The tongue cleaner according to claim 1, wherein said sheet is attached to both of the front and back faces of said head.

4. The tongue cleaner according to claim 1, wherein a woven textile or a nonwoven textile is used as said sheet.

5. The tongue cleaner according to claim 1, wherein said woven textile is one in which either weft yarns or warp yarns are mainly raised so that either the raised weft yarns or raised warp yarns of the woven textile are attached to either one or both of the front face and back face of said head in a direction intersecting with the longitudinal direction of said rod-shaped handle.

6. The tongue cleaner according to claim 1, wherein said woven textile is a pile-woven textile, in which pile yarns are used for the weft yarns or warp yarns of said pile-woven textile, said pile yarns being attached in a direction intersection with the longitudinal direction of said rod-shaped handle.

7. The tongue cleaner according to claim 1, wherein said head is configured such that said sheet is provided on a surface of a core member on which said concavely curved face is formed, said sheet being fixed at a side of said core member.

8. The tongue cleaner according to claim 7, wherein a gap is formed between said concavely curved face and said sheet opposite to said concavely curved face.

9. The tongue cleaner according to claim 1, wherein said head is configured such that said sheet is provided on a surface of a core member on which said convexly curved face is formed, said sheet being fixed at a side of said core member.

10. The tongue cleaner according to claim 1, wherein said head is configured such that said sheet covers a surface of a core member having a flat cross section, and a ring connected to said rod-shaped handle is fitted from outside into a side of said sheet at a side of said core member.
